(19) United States
(12) Patent Application Publication
(10) Pub. No.: US 2020/0101161 A1
(43) Pub. Date: Apr. 2, 2020

(54) Photosensitive Dyes and Method of Using Said Dyes

(71) Applicant: STRT LLC, Merion, PA (US)

(72) Inventors: Richard Titlebaum, Merion, PA (US); Som Tyagi, Garnet Valley, PA (US)

(73) Assignee: STRT LLC, Merion, PA (US)

(21) Appl. No.: 16/693,422

(22) Filed: Nov. 25, 2019

Related U.S. Application Data
(63) Continuation-in-part of application No. 16/199,267, filed on Nov. 26, 2018, now abandoned.
Application No. 16/199,267 is a continuation-in-part of application No. 15/538,227, filed on Jun. 21, 2017, now Pat. No. 10,136,963.
(60) Provisional application No. 62/095,168, filed on Dec. 22, 2014.

(51) Int. Cl. A61K 41/00; A61K 9/00; A61N 5/06; A61F 13/02

(52) U.S. Cl. CPC A61K 41/0057; A61K 9/0014; A61F 2013/00906; A61F 13/02; A61N 5/062

(57) ABSTRACT

A bandage and a method of treating a wound with the bandage is provided. The method includes the steps of applying a photosensitive dye to the wound; covering the wound with a bandage; and applying a light to the dye such that the dye generates a reactive oxygen species.

### CROSS-REFERENCE TO RELATED APPLICATIONS

The present application is a continuation-in-part application of U.S. patent application Ser. No. 16/199,267 (pending), filed on Nov. 26, 2018, which is a continuation-in-part application of U.S. patent application Ser. No. 15/538,227, filed on Jun. 21, 2017 and issued on Nov. 27, 2018 as U.S. Pat. No. 10,136,963, which is a 371 of PCT Application PCT/US2015/66985, filed on Dec. 21, 2015, which claims priority from U.S. Provisional Patent Application 62/095,168, filed on Dec. 22, 2014, which are all incorporated by references in their entireties.

### FIELD OF THE INVENTION

This invention relates generally to wound and other injury healing, and more particularly to a dye that can be applied to a wound or injury for photodynamic therapy and methods of using the dye.

### BACKGROUND OF THE INVENTION

Any wound, surface scrape or deeper, can potentially be colonized, and subsequently infected, by pathogenic and other bacteria, such as Methicillin-resistant Staphylococcus aureus (MRSA), which could delay healing time and/or lead to systemic infection. Athletes, daycare and school students, military personnel in barracks, and those who recently received inpatient medical care are at higher risk for MRSA. Currently there is no accepted prevention protocol other than good hygiene and reducing sharing of personal items such as towels and razors.

It would be beneficial to provide a bacteria reduction or elimination device to reduce the spread of bacteria.

### SUMMARY OF THE INVENTION

In accordance with one aspect of this invention there is provided a bactericidal application for medical treatment comprising a photosensitive dye being activatable to produce a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, when irradiated by light, the dye being selected from the group consisting of Gentian Violet, Crystal Violet, Toluidine Blue, Indocyanine Green, Xanthines, Rose Bengal, Fluorescein, Eosin, Erythrosine, Phenothiazines, Methylene Blue, Porphyrins, annatto extract, anthocyanins, B-carotene, beta APO 8 Carotenal, black currant, burnt sugar, Canthaxanthin, caramel, carbo medicinalis, carmine, carmine blue, carminic acid, carrot, chlorophyll, chlorophyllin, cochineal extract, copper chlorophyll, copper chlorophyllin, curcumin, curcumin/CU-chloro, grape, hibiscus, lutein, mixed carotenoids, paprika, riboflavin, spinach, stinging nettle, titanium dioxide, zinc oxide, turmeric, aronia/red fruit, beet juice, paprika extract, paprika oleoresin, amaranth, black PN, carmoisine, fast red E, erythrosine, green S, patent blue V, ponceau 4R, quinoline yellow, Red 2G, sunset yellow, tartrazine, Lake allura red, Lake amaranth, Lake brilliant blue FCF, Lake carmosine, Lake erythrosine, Lake indigo carmine, Lake ponceau 4R, Lake quinoline yellow, Lake sunset yellow, Tartrazine (FD&C Yellow No. 5) Curcumin, Riboflavin 5′-monophosphate sodium salt 441, Allura Red AC (FD&C Red No. 40) New Coccine (CI 16255, Food Red 7), Chromotrope FB (CI 14720, Food Red 3), Indigo Carmine (FD&C Blue No. 1), FD&C blue #2, Lissamine Green B Napthol Green or Acid Green Cochineal 530, Carmoisine azorubine 515, Amaranth 523, Brillant Scarlet 4R 503, Chlorophylls and copper complexes 633, Brillant black BN (PN), Chocolate Brown HT, Beta-carotene 470, Bixin Lycopene 530, Betanin Riboflavin 445, Riboflavin 5I-monophosphate sodium salt 441, Erythrosin B sodium salt, and teas.

Another aspect of this invention provides a method of treating a wound. The method comprises the steps of: applying a photosensitive liquid dye being activatable to produce a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, when irradiated by light to a wound; applying a bandage over the wound; and directing light to the bandage such that the light penetrates the bandage and activates the dye.

Still another aspect of this invention entails a method of producing a bactericidal bandage. The method comprises the steps of providing a bandage having a plurality of pores or interstices; and impregnating the bandage with a photosensitive dye so that the dye enters the pores or interstices.

In accordance with another aspect of the invention, a method of killing bacteria in a wound comprises the steps of: providing a dye-impregnated bandage having a plurality of pores or interstices, the bandage having been impregnated with a photosensitive dye so that said dye enters said pores or interstices; applying the dye-impregnated bandage over a wound; and irradiating the bandage with light to activate the photosensitive dye, whereupon the photosensitive dye produces a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, to act as a bactericide in wound to kill bacteria therein.

### BRIEF DESCRIPTION OF THE DRAWINGS

Other aspects, features, and advantages of the present invention will become more fully apparent from the following detailed description, the appended claims, and the accompanying drawings in which like reference numerals identify similar or identical elements. In the drawings:

FIG. 1 is an enlarged side elevation view of one exemplary bactericidal insert, e.g., a paper point, constructed in accordance with this invention for introduction into a canal in a tooth during a root canal procedure;

FIG. 2 is an enlarged view of the portion of the insert of FIG. 1 shown within the circle identified by the reference number 2;

FIG. 3 is a greatly enlarged illustration of a tooth, without its surrounding gum structure and bone structure, but shown in cross-section into which inserts like shown in FIG. 1 have been inserted into two prepared canals within the tooth, and showing the irradiation of the inserts via light applied from outside the tooth;

FIG. 3A is a plot of free radical concentration generated vs. irradiation time in a porous insert of FIG. 1 soaked with a 0.01% aqueous solution of methylene blue;

FIG. 4 is a block diagram showing one method in accordance with this invention for producing bactericidal inserts of this invention;

FIG. 5 is a block diagram showing two methods in accordance with this invention for killing bacteria within a canal in a tooth using an insert constructed in accordance with this invention;

FIGS. 6-8 constitute charts of tests conducted using components of the subject invention;

FIG. 9 is a perspective view of a bandage according to an exemplary embodiment of the present invention;

FIG. 10 is a perspective view of the bandage of FIG. 9 being applied to a wound and being irradiated with light;

FIG. 10A is a perspective view of a dye according to the exemplary invention applied to a wound and a bandage being applied to the wound;

FIG. 11 is a perspective view of the bandage of FIG. 9, with a flexible material incorporated therein;

FIG. 12 is a perspective view of a suture incorporating the dye of the present invention used to suture a wound;

FIG. 13 is a perspective view of a bandage according to the present invention with a light source incorporated therein;

FIG. 14 is a side elevational view of the dye according to the present invention applied to the interior of a digestive tract and being irradiated by an external light source;

FIG. 15 is a perspective view of a flexible material impregnated with the dye of the present invention;

FIG. 16 is a perspective view of a bandage with a removable portion placed over a wound;

FIG. 17 is a perspective view of the bandage of FIG. 16, with the removable portion pulled away to expose the wound to light; and

FIG. 18 is a perspective view of a dye with a liquid bandage over a wound.

### DETAILED DESCRIPTION OF THE PREFERRED EMBODIMENT

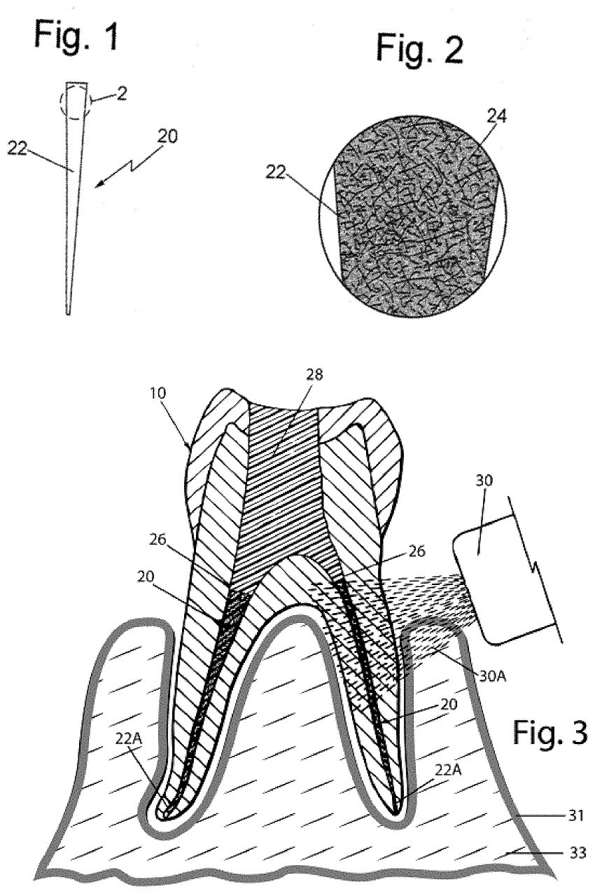

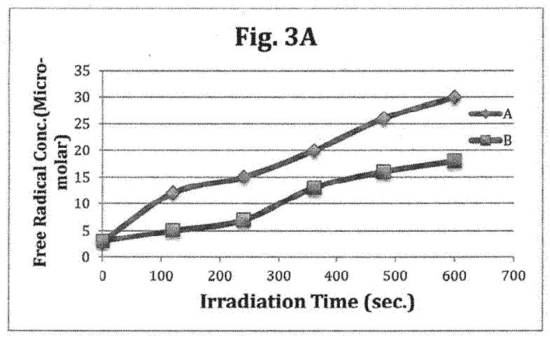

Referring now to the various figures of the drawing wherein like reference characters refer to like parts, there is shown at 20 in FIGS. 1 and 2 one exemplary embodiment of a bactericidal insert for introduction into a canal in a tooth 10 during a root canal procedure. The insert 20 basically comprises a carrier 22 which is porous and includes a photosensitive dye 24, (to be described later) which had been introduced into its pores or interstitial spaces and then dried. The insert is arranged to be deployed within the prepared canal 26 (FIG. 3) in the tooth with the photosensitive dye being wetted to produce a liquefied photosensitive dye, which is then activated by a light source external to the tooth to release a single or multiple reactive oxygen species, such as singlet oxygen, superoxide, hydroxyl, and nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O3), lipid peroxide (LOOH), peroxynitrite (ONOO—), hypochlorite (OCl—), hydroxyl ion, peroxide, into the canal to destroy any bacteria therein. In the embodiment shown the tooth has two roots so each root has an insert constructed in accordance with this invention deployed therein. It should be noted at this juncture that the location of the top end portion of each of the inserts 20 shown in FIG. 3 is for illustrative purposes only and may not represent the actual location in a particular patient's tooth.

As best seen in FIG. 1 in accordance with one exemplary preferred embodiment the carrier is in the form of a conventional “paper point”, like that used in traditional endodontic procedures and which is available from various manufacturers/suppliers. The carrier may take forms other than simple paper points, such as paper points with a solid metal core or metal impregnated in the paper, engineered points with nano-porous cavities made of gutta percha or some other material, nano-porous ceramic materials, sponges, etc., so long as the carrier is of a shape and construction which is capable of being inserted into a prepared canal 26 in a tooth and to extend sufficiently deeply within the canal so that the a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O3), lipid peroxide (LOOH), peroxynitrite (ONOO—), hypochlorite (OCl—), hydroxyl ion, and peroxide, released by its photosensitive dye can reach all portions of the canal. In this regard, preferably, the material of the carrier should be somewhat resistant to compression and deformation as the carrier is introduced into the canal to ensure that the carrier can reach the lowermost portions of the canal. Moreover, the carrier can be designed so that its pores or interstitial spaces are of a desired size to effectively hold the photosensitive dye therein.

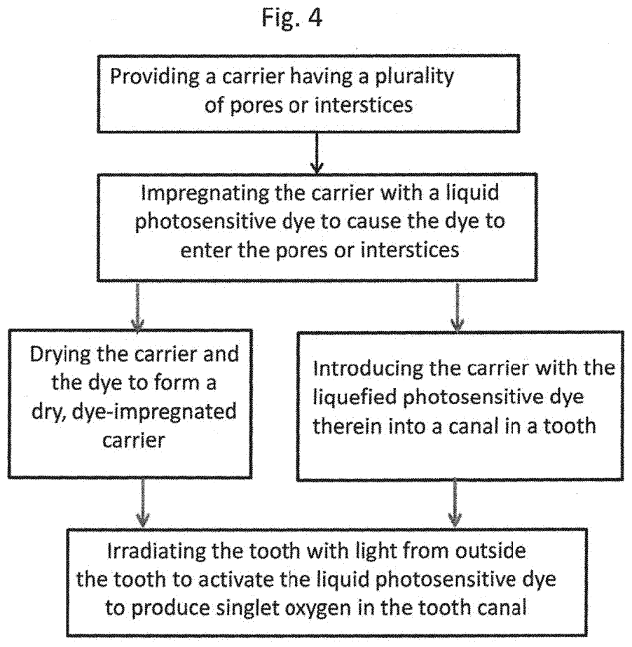

In accordance with one aspect of this invention and as shown in the flowchart of FIG. 4 the porous inserts 22 can be prepared by soaking them in the photosensitive dye 24. The dye-soaked inserts 20 may be washed to remove excess dye. The washing, if used, will leave a sufficient amount of the dye within the pores, and within the fibers of the paper point carrier, to be activated by the light to produce the single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, if the carrier is subsequently wetted and thereafter irradiated with light, as will be described later.

Still alternatively, instead of drying the carrier and the dye 24, the dye 24 may remain liquefied within the carrier for introduction of the carrier with the liquefied dye into the tooth canal 26 and then sealed into the tooth 10. The tooth 10 can be subsequently irradiated to activate the liquefied dye 24 to produce a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, in the tooth canal 26.

The carrier with the photosensitive dye therein, whether wet or dry, has sufficient resistance to axial compression so that the carrier can be deployed or introduced into the canal by inserting its pointed distal end 22A into the prepared tooth canal 26 and then tamped or otherwise pressed down the canal until its distal end reaches the bottom of the canal or as close to it as necessary. Once the insert 20 is in position, if dry, insert 20 can be wetted with any suitable wetting agent, e.g., a few drops of water or saline or an antibacterial solution, or wetting by the naturally occurring moisture in the tooth, whereupon the wetting agent/natural tooth moisture wicks down the length of the insert to release or re-liquefy the dried photosensitive dye. The liquid photosensitive dye can then diffuse into the surrounding tooth structure.

The inserts can then be sealed within the tooth with any suitable conventional filling 28, thereby sealing the inserts with the now liquid photosensitive dye within the tooth's canals. The tooth can then be exposed to suitable activating light 30A from outside of the tooth by a suitable light source 30 to activate the dye 24 (which is not only resident in the canal but has also diffused into the surrounding tooth structure), whereupon the dye 24 releases a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, thereby killing any bacteria to which the dye 24 is exposed. Moreover, since the liquid photosensitive dye will diffuse with time from the canal, the subject invention enables killing bacteria in a wider area than the area to which the photosensitive dye was initially delivered. Further still, since the insert is totally sealed within the tooth, staining of the adjacent teeth and gums does not result. Additionally, because the insert material is sealed within the tooth, reactive oxygen species will not adversely affect adjacent teeth and gums.

As shown in FIG. 3, activating light 30A can be shone directly on tooth 10, allowing the light to pass through tooth 10 and to insert 20 to activate the photosensitive dye within insert 20. Alternatively, activating light 30A can be shown on gum 31, passing through gum 31 and bone 33, as well as tooth 10 to insert 20 to activate the photosensitive dye within insert 20.

FIG. 3A is a plot of free radical concentration generated vs. irradiation time in a porous insert 22 of FIG. 1 soaked with a 0.01% aqueous solution of methylene blue and placed: [A] inside an extracted tooth as shown in FIG. 3, and [B] placed on the inner surface of a pig's jaw, such that the light is incident on the outer surface of pig's jaw and transmitted through gum, bone and tooth tissues. In both cases, the irradiation source is a red LED (630±10 nm) with an intensity of 310 Watts/m². While plot A shows a higher free radical concentration, plot B shows that application of an activating light against a gum/jawbone still transmits sufficient irradiation energy therethrough to generate a high free radical concentration for effective treatment.

It should be pointed out at this juncture that the means for irradiating the tooth with the activating light can be provided by any suitable means 30. For example, such means could comprise a small hand holdable illumination device, such as an LED laser, that is arranged to be inserted into the patient's mouth closely adjacent the tooth in issue. The patient himself/herself can hold that illumination device while in the dental chair to activate the photosensitive dye, thereby freeing the Dentist for other activities. In fact, it is contemplated that the patient may be provided with an illumination device to take home to use at some subsequent time to reactivate the photosensitive dye within the tooth and thereby kill any bacteria that may have avoided destruction upon the first application of the light therapy. Such application of the illumination device may be performed multiple times to reactivate the photosensitive dye each time that the illumination device is applied.

While the light source for activating the photosensitive dye of the insert has been described above as being a hand-held device, such as could be used by the patient, it could also be designed to adhere to gum tissue as a cover or sit on gum tissue or be designed as a modified cotton roll or other gauze, with a built-in light and power source.

In whatever form the light is manufactured, the light could also include some control circuitry to establish a preset amount of time and the times that the light operates so that patient compliance is unnecessary. The light could also be manufactured to keep a record of the amount of time that the light was turned on to corroborate reported patient compliance. In accordance with one preferred exemplary embodiment of the invention the photosensitive dye is selected from the group consisting of Gentian Violet, Crystal Violet, Toluidine Blue, Indocyanine Green, Xanthines (e.g., Rose Bengal, Fluorescein, Eosin, Erythrosine, etc.) Phenothiazines (e.g., Methylene Blue), Porphyrins, etc. Those dyes when in liquid form and irradiated by light of an appropriate wavelength, e.g., a wavelength near the maximum absorption of the dye being used, produce free radicals. In particular, usually a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, is produced, but other free radical species have been reported. Those free radicals have a deleterious effect on bacterial cells. While the host cells may also subject to the deleterious effects of the free radicals, it appears that the number of free radicals necessary to destroy the bacterial cells is lower than the amount that would cause appreciable harm to host cells. This concept is known as the Therapeutic Index. Moreover, the photosensitive dye and light combination as delivered by this invention is effectively targeted since the dye and light combination is delivered in a confined location, namely, within the tooth canal and thus does not have to be delivered systemically.

In lieu of, or in addition to, the photosensitive dyes described above, other dyes, such as, for example, annatto extract, anthocyanins, B-carotene, beta APO 8 Carotenal, black currant, burnt sugar, Canthaxanthin, caramel, carbo medicinalis, carmine, carmine blue, carminic acid, carrot, chlorophyll, chlorophyllin, cochineal extract, copper chlorophyll, copper chlorophyllin, curcumin, curcumin/CU-chloro, grape, hibiscus, lutein, mixed carotenoids, paprika, riboflavin, spinach, stinging nettle, titanium dioxide, zinc oxide, turmeric, aronia/red fruit, beet juice, paprika extract, paprika oleoresin, amaranth, black PN, carmoisine, fast red E, erythrosine, green S, patent blue V, ponceau 4R, quinoline yellow, Red 2G, sunset yellow, tartrazine, Lake allura red, Lake amaranth, Lake brilliant blue FCF, Lake carmosine, Lake erythrosine, Lake indigo carmine, Lake ponceau 4R, Lake quinoline yellow, Lake sunset yellow, Tartrazine (FD&C Yellow No. 5) Curcumin, Riboflavin 5′-monophosphate sodium salt 441, Allura Red AC (FD&C Red No. 40) New Coccine (CI 16255, Food Red 7), Chromotrope FB (CI 14720, Food Red 3), Indigo Carmine (FD&C Blue No. 1), FD&C blue #2, Lissamine Green B Napthol Green or Acid Green Cochineal 530, Carmoisine azorubine 515, Amaranth 523, Brillant Scarlet 4R 503, Chlorophylls and copper complexes 633, Brillant black BN (PN), Chocolate Brown HT, Beta-carotene 470, Bixin Lycopene 530, Betanin Riboflavin 445, Riboflavin 5I-monophosphate sodium salt 441, Erythrosin B sodium salt, teas, and/or juices, can also be applied to the subject tooth to generate a single or multiple reactive oxygen species.

If desired, the insert may include a radio-opaque material or tag. For example, the insert with its tagged porous/nanoporous material and photosensitive dye would be sealed in place and the tooth can be restored on top of the insert. This would be much faster and less technically challenging than current or past techniques. Moreover, the insert with the radio-opaque tag could be readily identified by radiograph to verify its presence and to provide an indication of the type of antibacterial treatment that was performed. With current technologies, one can determine by x-ray or other examination that a root canal treatment has been completed, but one would have absolutely no idea what protocol, if any, was used to attempt to kill the bacteria in the tooth during the procedure.

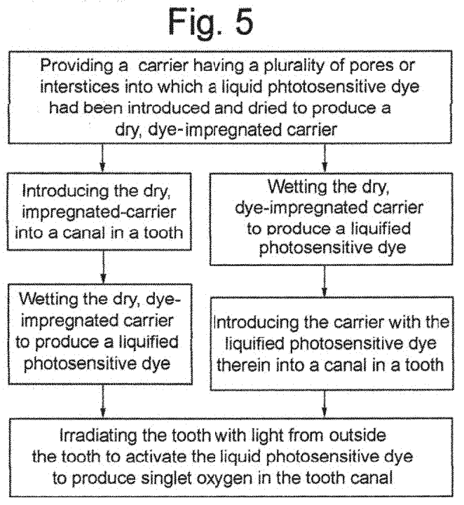

Turning now to FIG. 5 the details of two methods of destroying bacteria in a root canal will now be described. One method shown by the sequence of events on the left side of the flow diagram basically comprises providing a dry dye-impregnated carrier having a plurality of pores or interstices which carrier had been impregnated with a liquid photosensitive dye so that the dye entered the pores or interstices. The carrier with the liquid photosensitive dye therein had been dried to form the dry dye-impregnated carrier.

The dry dye-impregnated carrier is introduced into the canal in the tooth to a desired position. When said dry, dye-impregnated carrier is in that desired position a portion thereof is located adjacent the surface of the tooth contiguous with the canal. The dry dye-impregnated carrier is then wetted by either the use of a wetting agent or by the natural moisture within the tooth canal, whereupon said moisture/wetting agent wicks through the dry dye-impregnated carrier to produce liquefied photosensitive dye and the insert is then sealed within the tooth by any conventional sealing technique. After that has been accomplished the tooth is irradiated with light from outside the tooth to activate the liquefied photosensitive dye within the canal, whereupon the liquid photosensitive dye produces a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, to act as a bactericide in the canal of the tooth to kill the bacteria therein.

As an alternative to the method described immediately above for destroying bacteria in a root canal procedure, and as shown by the sequence of events shown on the right side of the flow diagram the dry dye-impregnated carrier is wetted prior to introduction into the canal of the tooth, e.g., the carrier is wetted with a wetting agent to produce the liquefied photosensitive dye, and that rewetted carrier is introduced into the canal of the tooth. The insert with the wetted carrier may be provided in a sealed package, which can be opened when needed and the wetted insert removed from the package and then introduced into the prepared canal.

Alternatively, the insert can be provided in a dry state and then wetted prior to deployment into the prepared canal. In any case, once the wetted insert is within the canal and is sealed therein, such as described above, the tooth can then irradiated with light from outside the tooth to activate the liquefied photosensitive dye within the canal, whereupon the liquid photosensitive dye produces a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, superoxide, hydroxyl, nitric oxide and others to act as a bactericide in the canal of the tooth to kill the bacteria therein.

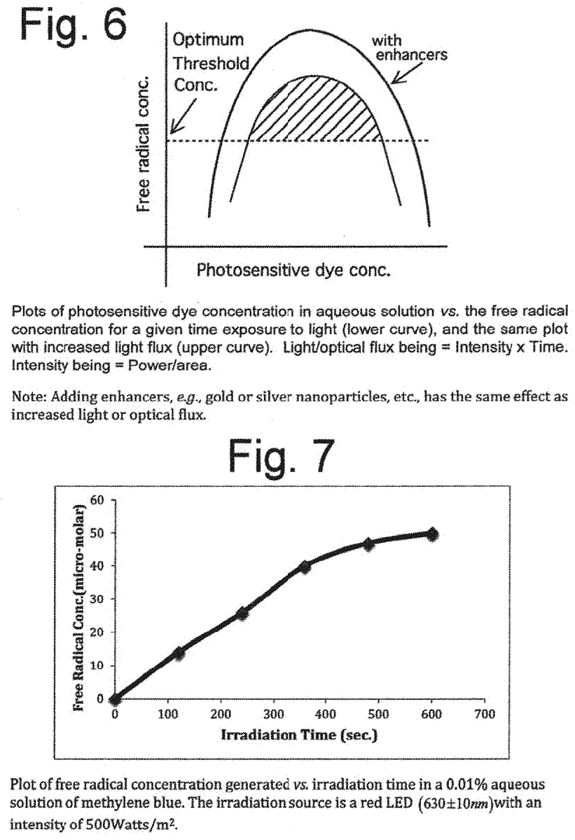

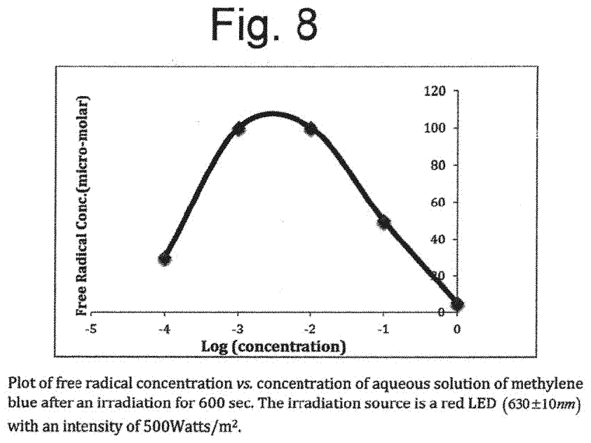

It should be pointed out at the juncture that the inventors have carried out some preliminary in vitro tests of carriers and some photo sensitive dyes like those used in the subject invention. The charts shown in FIGS. 6-8 represent some preliminary findings indicating the efficacy of the release of a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, when subjected to suitable light for activating the photosensitive dyes.

As should be appreciated by those skilled in the art from the foregoing the subject invention provides a viable means for ensuring that all bacteria are killed by a root canal procedure making use of an insert and methods in accordance with this invention. Even if all bacteria are not killed by the initial application of light, with the insert remaining sealed within the tooth's canal additional exposures to light from outside the tooth can be accomplished thereafter to effectively destroy any residual bacteria.

Moreover, the subject invention can decrease “chair time” for the Dentist as the activating light can be delivered without the patient being in the chair. In fact the activating light can be delivered outside of the Dentist's office. The activating light can be delivered for an extended period of time and can be reapplied by the patient if there is pain or swelling during post-operative period or if infection recurs at some future time. In this regard if bacteria or infection persist or return at some point after treatment, the subject invention provides the ability for the patient to have the tooth irradiated again, noninvasively, and potentially killing any residual bacteria at this later date. Additionally, the activating light can be periodically repeatedly applied to the tooth, such as, for example, during semiannual dental checkups, thereby reactivating the dye during each repeated light application. It should be emphasized that the creation of the singlet oxygen species by photo-excitation is a completely reversible phenomenon. This means that the efficacy of the dye to produce singlet oxygen species does not diminish with repeated photoexcitation.

It should be appreciated also that the activating light does not necessarily have to be delivered at the time of insertion of the dye-impregnated carrier into the tooth canal, but instead, can be delivered at a later date, and at a location outside the Dentist's office, such as, for example, by self-radiating by the patient in the patient's home.

Currently the only methods to deal with recurrent infection are invasive and involve either redoing the root canal treatment, a surgical treatment of the root and the surrounding infection, or an extraction. The subject invention should spare the patient these invasive treatments.

The following table represents the advantages of the PDT treatment in root canal procedures as provided by the subject invention over prior art PDT treatments.

| | PDT of the subject |
|---|---|
| Prior Art PDT | Invention |
| Liquid | Porous/nanoporous with material |
| | dye incorporated |
| Entire treatment in Dental | Once sealed in tooth, no Dental |
| office, taking up patient and | office time needed |
| Light/# of photons limited to | Light/# of photons not limited |
| size of fiber that can get into | |
| Invasive for repeat treatment | Non-invasive for repeat treatment |
| No thought about | Ideal concentration determined |
| Narrow spectrum of light | Wide spectrum of light |
| Light into canal | Light from outside with either a |
| | hand-held light source or a device |

Accordingly, the subject invention provides a means and method to disinfect a root canal with the potential for future disinfection that can replace current filling materials/techniques used in root canal procedures. Moreover, if necessary to retreat or re-enter the root canal space, the insert of this invention could be removed easier and faster than anything that is currently used. Further still, if desired the carrier may contain photosensitive dye and any type of enhancer, such as but not limited to, ethylenediaminetetraacetic acid (EDTA), nanoparticles of gold or silver, quantum dots, graphene, sodium ascorbate, etc.

It must be noted at this time that the subject invention can be used for other dental applications than root canal procedures. For example, a photosensitive dye, like those described above, can be incorporated into a carrier that can be packed into a periodontal pocket, then activated with an external light. The carrier can be removed at a later date after periodontal pocket has been sufficiently disinfected/sterilized. Alternatively, the carrier can be designed to absorb over time.

In fact, the subject invention is not limited to dental applications. In this regard, the subject invention may be used as a means of wound protection or wound therapy. For example, as described in more detail below, an enhanced antibacterial bandage can be constructed in accordance with this invention having a carrier, e.g., a porous gauze, holding the photosensitive dye and which can be activated with an external light to decrease wound healing time, prevent MRSA and other infections, prevent cross-infection, provide skin disinfection prior to surgery. Such devices could decrease post-operative infection and reduce hospital readmissions, decrease mortality, decrease cost to the patient, the hospital and the insurance company. Such a device could also provide post-surgical wound protection and suture site protection. It is contemplated that those bandages may also be designed with light built into the gauze and with its own power supply so that patient compliance is unnecessary.

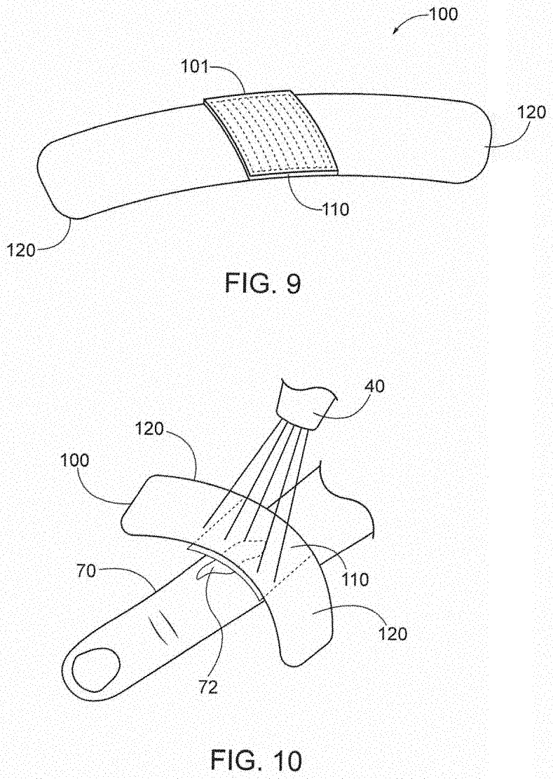

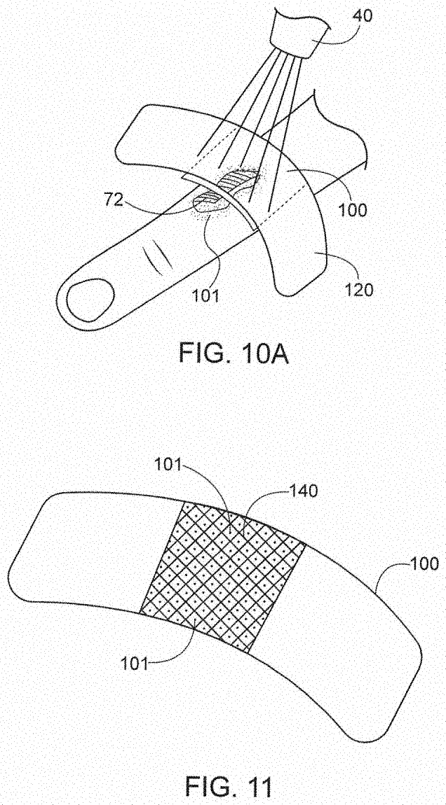

Referring to FIGS. 9 and 10, a dressing or bandage assembly according to an exemplary embodiment of the present invention is shown. An individual can place a photosensitive dye 101 and a bandage 100, or bandage 100 with photosensitive dye 101 incorporated therein that releases the photosensitive dye 101 when moist, over a wound. Dye 101 can be a liquid, a flexible or inflexible solid, a gel, or other suitable form for applying to a wound. Subsequently the individual would introduce light to the bandage 100 such that the light passes through the bandage 100, activates the photosensitive dye 101, causing the production and release of reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, superoxide, hydroxyl, nitric oxide and others, which kill bacteria. The light can be a device, sunlight, or even indoor light. The individual can continue with his/her activities and not stop for treatment after the application of the bandage 100 in some cases. Bacteria cannot become resistant to the photodynamic therapy. The light treatment, resulting in the production of reactive oxygen species that can kill bacteria, can be repeated as often as prescribed, desired or needed without adding new photosensitive dye 101 or a new bandage 100, although it may be desired to remove and replace bandage 100 for other treatment reasons.

Bandage 100 can be transparent, translucent, or partially transparent to light. Bandage 100 can include a non-adhesive area 110 that is to be applied directly over a wound, such as a cut, a burn, an abrasion, a scrape, or other type of wound. Adhesive portions 120 can be located on either side of or around all sides of non-adhesive area 110 so that bandage 100 can be directly applied to a skin 70 with non-adhesive area 110 directly over a wound 72 and adhesive portions 120 can be applied to the skin 70 on either side of wound 72.

Optionally, dye 101 can be applied to healthy tissue surrounding a wound, a pre-operative site, or other non-wound area to reduce or eliminate bacteria at the dye-applied tissue.

Alternatively, adhesive portions 120 can be omitted in their entireties and bandage 100 can be wrapped around the skin 70 so that part of bandage 100 is over the wound 72, and bandage can be secured to the skin 70, such as with medical tape. Still alternatively, a self-adhesive bandage, such as CoBan™ wrap can be used, with or without photosensitive dye 101 incorporated therein. In any case, a light source 40 is applied to bandage 100 to activate photosensitive dye 101 at the site of wound 72.

In one embodiment, as shown in FIG. 10A, bandage 100 has no photosensitive bactericidal dye 101 incorporated with bandage 100. Prior to applying bandage 100 to wound 72, a photosensitive bactericidal dye 101, such as any of the dyes disclosed above, can be applied in and around wound 72. Dye 101 can be applied as a liquid or gel, and can be sprayed on, dropped on, applied with a brush, or other suitable application method. The dye 101 can be liquid or, alternatively, the dye 101 can be solid, such as in powder form. The solid dye 101 can be wetted with a wetting agent after applying the dye 101 to the wound, such as with water or some other wetting agent sprayed onto or otherwise applied to the dye 101, or, alternatively, natural body fluids from the wound 72 can be used to wet the dry dye 101. Optionally, bandage 100 can be provided as part of a package or kit that includes a wetting agent. This allows bandage 100 to be used in the field where water or other external wetting agent may not be otherwise available.

Bandage 100 can include a plurality of pores or interstices formed therein wherein the dye 101 is impregnated into bandage 100 so that the dye 101 enters the pores or interstices. Optionally, as shown in FIG. 11, a flexible material 140, such as silicone, can be incorporated into bandage 100 such that the dye 101 is incorporated into the flexible material 140. In either of the above embodiments, the dye 101 can be a solid or a liquid.

As shown in FIGS. 10 and 10A, a wound 72 can be treated using bandage 100 by applying a photosensitive dye 101 to wound 72, applying bandage 100 over wound 72, and directing light to bandage 100 such that the light penetrates bandage 100 and activates the dye 101, generating a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, at wound 72 to kill bacteria therein. The dye 101 can be wetted either prior to applying bandage 100 over wound 72 or, alternatively, the dye 101 can be wetted after applying bandage 100 over wound 72 by wetting bandage 100 and allowing the wetting agent to soak through bandage 100 to the dye 101.

Alternatively, the wound 72 can be treated using bandage 100 by applying a photosensitive dye impregnated bandage 100 to wound 72, applying bandage 100 over wound 72, and directing light to bandage 100 such that the light penetrates bandage 100 and activates the dye 101 in bandage 100, generating a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, at wound 72 to kill bacteria therein. The dye 101 can be wetted by natural moisture from wound 72, by wetting the dye 101 prior to applying bandage 100 over wound 72 or, by wetting the dye 101 after applying bandage 100 over wound 72 by wetting bandage 100 and allowing the wetting agent to soak through bandage 100 to the dye 101.

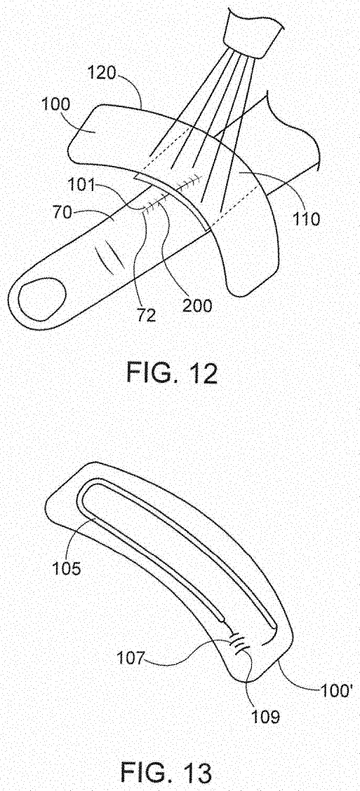

In an alternative embodiment, instead of impregnating bandage 100 with the dye 101, the dye 101 can be applied to a suture 200, shown in FIG. 12. Suture 200 can be an inner suture, an outer suture, or both. Suture 200 is a thread that has dye 101 applied to it. Dye 101 can be applied to the exterior of the thread or, alternatively, dye 101 can be impregnated or manufactured into the thread.

After using suture 200 to suture a wound, such as wound 72, suture 200 and the wound 72 can be wetted and then irradiated with a light, activating the photosensitive dye 101 in suture 200, thereby generating a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, at wound 72 to kill bacteria therein. The wetting process can be performed using natural body fluids, such as blood, or other fluids, from wound 72 or, alternatively, by applying a wetting agent to the wound 72 and suture 200 prior to irradiating the wound 72. Optionally, bandage 100 can be applied over the sutured wound 72.

Alternatively, instead of bandage 100 being at least partially transparent to light, a bandage 100′, shown in FIG. 13, can be provided that is opaque to light. Bandage 100′ includes a light source 105 incorporated therein. Light source 105 can be a single elongate light or a plurality of lights strung together. A power supply in the form of a battery 107 is provided and can be attached to an exterior of bandage 100′. Also, an on/off switch 109 can be electrically attached to battery 107 to power light source 105 on or off. Dye 101 applied to the wound 72 or incorporated into bandage 100′ can be wetted by natural moisture from wound 72. Alternatively, bandage 100′ can be wetted from the exterior.

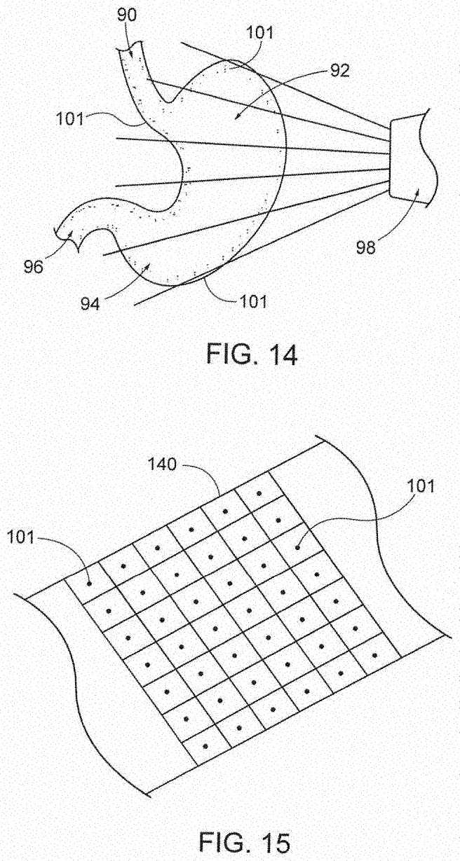

As shown in FIG. 14, dye 101 can be used without a solid carrier, such as bandage 100 or suture 200, and can be dissolved in a fluid to be swallowed or otherwise consumed by a patient for treating a digestive tract or any other body part, such as for killing bacteria. By way of example only, the consumed fluid with dye can coat an esophagus 90, stomach 92, pylorus 94, and duodenum 96 of the patient. An outside light source 98 is directed from exterior of the patient to the coated body parts, generating a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, on the interiors of these organs and killing bacteria that can cause stomach ulcers.

FIG. 15 shows an alternative embodiment of the dye 101 being impregnated within flexible material 140, such that the dye-impregnated material 140 can be applied to the surface of or integrated into, a common household device, such as a table, a doorknob, a cutting board, a cell phone case, a paper towel dispenser, a soap dispenser, a pen, a railing, a seat, a toilet seat, or any other common device that is frequently touched and can easily transmit bacteria, germs, and other harmful contagions between people. As discussed above, the material 140 can be silicone, although those skilled in the art will recognize that the material 140 can be other flexible materials. The dye-impregnated flexible material 140 can be applied to the exterior of the surface in a very thin coating. Alternatively, for materials with which material 140 can be intermixed, the dye-impregnated material 140 can become part of the surface to be treated.

To activate the dye, the surface 140 can be wetted, such as with water or a damp towel, and then exposed to light, such as sunlight, room lighting, or other ambient lighting. The exposure of the wet material 140 to the light activates the dye, generating a single or multiple reactive oxygen species, such as singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide, on the surface and killing bacteria on the surface.

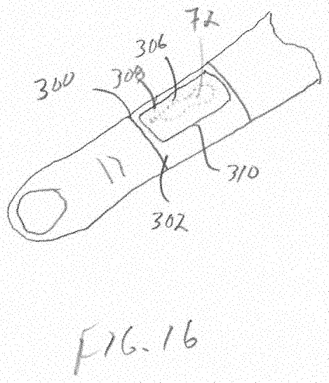

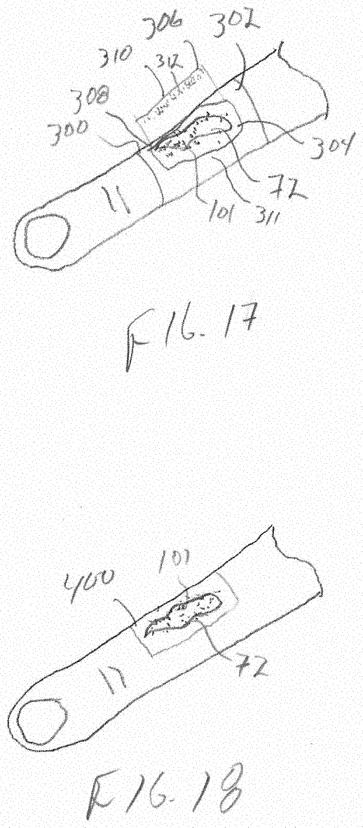

In an alternative embodiment, shown in FIGS. 16 and 17, a bandage 300 can have an opaque portion 302 that extends around wound 72, with a transparent or translucent window 304 surrounded by opaque portion 302 that can allow light to penetrate window 304 and activate dye 101. Window 304 can be covered by a removable opaque cover 306 that can be alternately placed over window 304 and wound 72 when it is not desired to activate dye 101 (as shown in FIG. 16) and removed from window 304 so that dye 101 can be exposed to light and activated (as shown in FIG. 17).

Cover 306 can have a fixed end 308 that is fixedly attached to bandage 300 adjacent to window 304 and a free end 310 that can be removably attached to bandage 300 on a distal side 311 of window 304. Free end 310 can have an adhesive 312 that can releasably engage bandage 300. Alternatively, free end 310 can have one part of a hook and loop combination while distal side 311 has the other of the hook and loop combination. Those skilled in the art will recognize that other releasable adhesives can also be used.

Still alternatively, as shown in FIG. 18, a liquid bandage 400 can be applied over wound 72. Dye 101 can first be applied to wound 72, and then liquid bandage 400 can be applied in liquid form over wound 72 and allowed to dry. Liquid bandage 400 dries to a transparent or translucent form so that light can penetrate liquid bandage 400 to activate dye 101. After drying, liquid bandage 400 can be covered by an opaque bandage (not shown) or covered with the patient's clothing (not shown).

Alternatively, instead of first applying a separate dye 101 and then the liquid bandage 400, the dye 101 can be incorporated into the liquid bandage 400 and dye 101 and liquid bandage 400 can be applied simultaneously. After drying, liquid bandage 400 with impregnated dye 101 can be covered by an opaque bandage (not shown) or covered with the patient's clothing (not shown).

Without further elaboration the foregoing will so fully illustrate our invention that others may, by applying current or future knowledge, adopt the same for use under various conditions of service.

We claim:

1. A method of treating a wound comprising the steps of:
(a) applying a photosensitive dye to the wound;
(b) covering the wound with a bandage;
and
(c) applying a light to the dye such that the dye generates a reactive oxygen species.

2. The method according to claim 1, further comprising, between steps (b) and (c), the step of wetting the wound.

3. The method according to claim 1, wherein step (b) comprises using an opaque bandage and wherein the method further comprises, between steps (b) and (c), the step of moving at least part of the bandage away from the wound.

4. The method according to claim 3, further comprising, after step (c), re-covering the wound with the bandage.

5. The method according to claim 1, wherein steps (a) and (b) are performed in a clinical environment and wherein step (c) is performed outside of the clinical environment.

6. A method of bandaging a wound comprising the steps of:
(a) applying a photosensitive dye to the wound;
(b) covering the wound with a bandage;
(c) wetting the dye; and
(d) applying a light to the dye such that the dye generates a reactive oxygen species.

7. The method according to claim 6, wherein the dye comprises a liquid.

8. The method according to claim 6, wherein the dye comprises a solid.

9. The method according to claim 6, wherein the dye is incorporated into the bandage and wherein steps (a) and (b) are performed simultaneously.

10. The method according to claim 6, wherein step (c) comprises wetting the dye from natural fluid from the wound.

11. The method according to claim 6, wherein step (c) comprises wetting the dye from an external fluid source.

12. The method according to claim 6, wherein step (d) comprises applying the light from outside of the bandage.

13. The method according to claim 6, wherein a light is incorporated into the bandage and wherein step (d) comprises applying light to the dye from the light.

14. The method according to claim 6, wherein the dye is selected from the group consisting of: Gentian Violet, Crystal Violet, Toluidine Blue, Indocyanine Green, Xanthines, Rose Bengal, Fluorescein, Eosin, Erythrosine, Phenothiazines, Methylene Blue, Porphyrins, annatto extract, anthocyanins, B-carotene, beta APO 8 Carotenal, black currant, burnt sugar, Canthaxanthin, caramel, carbo medicinalis, carmine, carmine blue, carminic acid, carrot, chlorophyll, chlorophyllin, cochineal extract, copper chlorophyll, copper chlorophyllin, curcumin, curcumin/CU-chloro, grape, hibiscus, lutein, mixed carotenoids, paprika, riboflavin, spinach, stinging nettle, titanium dioxide, zinc oxide, turmeric, aronia/red fruit, beet juice, paprika extract, paprika oleoresin, amaranth, black PN, carmoisine, fast red E, erythrosine, green S, patent blue V, ponceau 4R, quinoline yellow, Red 2G, sunset yellow, tartrazine, Lake allura red, Lake amaranth, Lake brilliant blue FCF, Lake carmosine, Lake erythrosine, Lake indigo carmine, Lake ponceau 4R, Lake quinoline yellow, Lake sunset yellow, Tartrazine (FD&C Yellow No. 5) Curcumin, Riboflavin 5′-monophosphate sodium salt 441, Allura Red AC (FD&C Red No. 40) New Coccine (CI 16255, Food Red 7), Chromotrope FB (CI 14720, Food Red 3), Indigo Carmine (FD&C Blue No. 1), FD&C blue #2, Lissamine Green B Napthol Green or Acid Green Cochineal 530, Carmoisine azorubine 515, Amaranth 523, Brillant Scarlet 4R 503, Chlorophylls and copper complexes 633, Brillant black BN (PN), Chocolate Brown HT, Beta-carotene 470, Bixin Lycopene 530, Betanin Riboflavin 445, Riboflavin 5I-monophosphate sodium salt 441, Erythrosin B sodium salt, and teas.

15. The method according to claim 6, wherein the reactive oxygen species comprises at least one of singlet oxygen, superoxide, hydroxyl, nitric oxide, free radicals, non-radicals, Hydroxyl radical (.OH), superoxide anion (.O₂—), lipid peroxyl (.LOO—), thiyl (.RS), hydrogen peroxide (H₂O₂), ozone (O₃), lipid peroxide (LOOH), peroxynitrite (ONOO—), hypochlorite (OCl—), hydroxyl ion, and peroxide.

16. A method of bandaging a wound comprising the steps of:
(a) applying a photosensitive dye to the wound;
(b) covering the wound with a bandage;
(c) wetting the dye; and
(d) generating a reactive oxygen species from the dye.

17. The method according to claim 16, wherein, prior to step (a), the dye is applied to the bandage.

18. The method according to claim 16, wherein step (c) comprises wetting the dye with fluid from the wound.

19. The method according to claim 16, wherein step (d) comprises generating singlet oxygen.

20. The method according to claim 16, wherein step (d) comprises exposing the dye to ultraviolet light.
